(19) United States
(12) Patent Application Publication
(10) Pub. No.: US 2024/0144800 A1
(43) Pub. Date: May 2, 2024

(54) ENVIRONMENT MONITORING AND CONTROL SYSTEM

(71) Applicant: SBT IP LLC, Miami, FL (US)

(72) Inventor: STEVEN MARIN, MIAMI, FL (US)

(73) Assignee: SBT IP LLC, MIAMI, FL (US)

(21) Appl. No.: 17/979,448

(22) Filed: Nov. 2, 2022

(51) Int. Cl. G08B 17/10; G01N 33/00

(52) U.S. Cl. CPC G08B 17/10; G01N 33/0063; G01N 33/0075; G08B 25/00

(57) ABSTRACT

An an environment monitoring and control system used to monitor public closed room facilities for the detection of a public nuisance or of a gas particle that can be identified as a controlled substance. The system comprises of a detector that has particle sensors, an array of chemical sensors, a motion sensor, a sound sensor, a temperature sensor, and a humidity sensor. The detector has a detector processor that records the signals received from the sensors and it is configured to communicate with either a personal handheld device or a central processing system. The central processing system of the system is configured to interpret the signals received from the detector and to send an alarm to any handheld device connected to the system. The central processing system has a central processor database that it uses to interpret the signals received from the detector.

### BACKGROUND

The present invention is directed to an environment monitoring and control system used to monitor public closed room facilities.

The inventors of the present invention have a background with schools and the dangers that occur in unmonitored facilities, such as restrooms.

Unmonitored facilities also lend themselves to areas wherein illicit drug behavior can occur, such as vaping or the smoking of marijuana, crack, cocaine, crystal meth, or other undetermined smokeable drug.

The use of illicit drugs in public restrooms by students is a nuisance that the inventors of the present invention wish to prevent.

The inventors also wish to prevent the bullying that typically occurs in the public restrooms.

The present invention was conceived to prevent the illegal use drugs in public restrooms and to prevent bullying that typically occurs in the public restrooms.

The present invention eliminates the need of having to have an actual person present or near the public restrooms to monitor the activity that occurs in the public restrooms by providing an adaptive sensor system that communicates with a central processing system when it detects particles or sounds that are deemed a nuisance or an illegal substance.

### SUMMARY

The present invention is an environment monitoring and control system used to monitor public closed room facilities for the detection of a public nuisance or of a gas particle that can be identified as a controlled substance.

The system comprises of a detector that has particle sensors, an array of chemical sensors, a motion sensor, a sound sensor, a temperature sensor, and a humidity sensor. The detector has a detector processor that records the signals received from the sensors and it is configured to communicate with a personal handheld device or a central processing system. The central processing system (hereinafter referred as a CPS) is configured to interpret the signals received from the detector and to send an alarm to any handheld device connected to the system. The CPS has a database that it uses to interpret the signals received from the detector. The database has known values for sounds, particles, chemical, and movement that are deemed of interest. If a value is deemed of interest, then the central processor is configured to send an alarm signal to the handheld device that is configured to receive the alarm.

The CPS of the present invention is also configured to communicate with a government database to update the known parameters for sounds, chemical, and particles identified by the government as being of interest. The CPS is also configured to send the government database updated parameters of unknown sounds and particles recorded in the closed room facilities.

An object of the present invention is to provide a system that may prevent bullying, loud sounds, and vandalism in closed room facilities, such as school public restrooms.

Another object of the present invention is to provide a system that will prevent the use of vaporable or smokeable drugs in closed room facilities.

Yet another object of the present invention is to provide a system that will identify new vaporable compounds used in public room facilities and then send them to a government database that can determine whether the particles are of interest.

Still yet another object of the present invention is to provide a system that uses artificial intelligence to update itself when new sounds or particles are detected by the detector.

### BRIEF DESCRIPTION OF THE DRAWINGS

These and other features, aspects, and advantages of the present invention will become better understood with regards to the following description, appended claims, and drawings where:

FIG. 1 shows a diagram of the present invention.

### DESCRIPTION

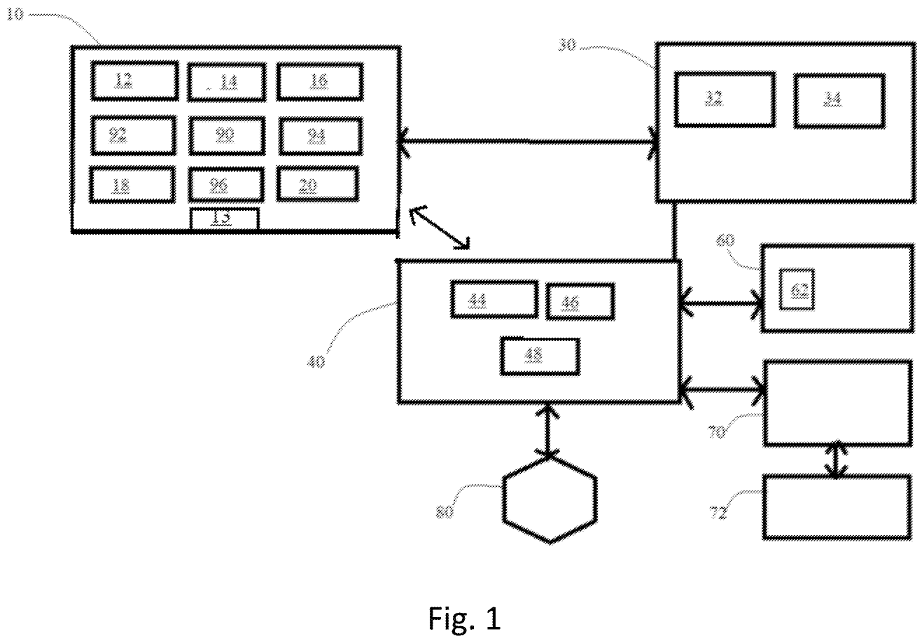

As seen in FIG. 1, an environment monitoring and control system used to monitor a closed room facility for the detection of a public nuisance or a gas particle that can be identified as a controlled substance.

The system comprises a detector 10 that has particle sensors 12, an array of chemical sensors 13, a motion sensor 14, a sound sensor 16, a detector communication device 18, a detector processor 20 that is configured to record any particle, chemical, movement, or sound sensed, hereinafter collectively referred as a recorded value, by either the particle sensor 12, the array of chemical sensors 13, the motion sensor 14, or sound sensor 16 and to transmit the recorded value to either a personal handheld device 30, a local police authority 60, or a central processing system 40 via the detector communication device 18 of the detector 10, the detector 10 is configured to be placed in the closed room facility. The central processing system 40 that is configured to interpret the recorded value received from the detector 10, a communication module 44 that is configured to communicate with either the detector 10, the personal handheld device 30, the local police authority 60 or a governmental database 70, the central processing system 40 has a central processor database 46, the central processing system 40 is configured to use the central processor database 46 to interpret the recorded value received from the detector 10 and to determine if the recorded value is of interest, the central processor database 46 has a plurality of known values for sounds, chemical, particles, and movement that are deemed of interest, if the recorded value is deemed of interest, then the central processing system 40 is configured to send an alarm to either the detector 10, the personal handheld device 30, the local police authority 60, or the governmental database 70, the central processing system 40 has an artificial intelligence module 48 that is configured to determine if the recorded value is new, and if so, then to add the recorded value to the central processor database 46, the artificial intelligence module 48 is also configured to scour an internet 80 to determine if the recorded value is of interest, and if so, then to identify the recorded value as a value that is of interest. And, a personal handheld device 30 that has a handheld device processor 32 and a transceiver 34, the personal handheld device 30 is configured to communicate with the detector 10 and the central processing system 40.

In an embodiment of the present invention, the system comprises the governmental database 70 that is configured to receive the recorded value, if it is new, and to send the recorded value to a governmental monitoring system 72 that is configured to determine if the recorded value is of interest, if so, then to update the governmental database 70 with the governmental value and to send the central processing system 40 with a new value deemed of interest.

In another embodiment of the present invention, the system comprises the local police authority, the local police authority 60 has a communication module 62 that is configured to receive alarms from either the central processing system 40 or the detector 10.

In yet another embodiment of the present invention, the detector comprises of a humidity sensor 90 and a temperature sensor 92, the detector 10 is configured to transmit any value collected from either the humidity sensor 90 or the temperature sensor 92 to the central processing system 40.

In an embodiment of the present invention, the particle sensor measures particles in a range that is between 1.0 μm to 10.0 μm. The particle sensor identifies particles that are contained in a vapor.

In a further embodiment of the present invention, the array of chemical sensors identify carbon monoxide, methane, ethanol, propane, isobutane, hydrogen, hydrogen sulfide, ammonia, nitric oxide, and nitrogen dioxide.

In another embodiment of the present invention, the detector comprises a vibration sensor 94, the detector 10 is configured to transmit any value collected from the vibration sensor 94 to the central processing system 40. The vibration sensor has the ability to detect vibrations or movements that happen to it: this allows a monitor of the detector to detect is someone is trying to damage the detector, move the detector, or if no record any vibration coming from the surface in which the detector is mounted or placed.

In still another embodiment of the present invention, the detector 10 comprises of a detector module 96 that is configured to store values that correspond to a value deemed of interest and to transmit the value deemed of interest to either the personal handheld device 30, the local police authority 60, or the central processing system 40.

The detector module uses algorithms to detect the following elements or compounds present in an environment: electronic cigarettes (Vape); cigarettes; and marijuana. The air quality in an environment is determined by measuring the volatile organic compounds in an environment and identifying the gasses harmful to health present in the environment.

Sounds that may be deemed of interest by the detector module 96 of the present invention are as follows: words deemed of interest; gun shots; screams (noise above a certain decibel); fire alarms; and glass breaking.

In preferred embodiments of the present invention, the detector communication device 18 of the present invention can be either a transceiver or an ethernet communication device.

An advantage of the present invention is that it provides a system that prevents bullying, loud sounds, and vandalism in closed room facilities, such as school public restrooms.

Another advantage of the present invention is that it provides a system that prevents the use of vaporable or smokeable drugs in closed room facilities.

Yet another advantage of the present invention is that it provides a system that identifies new vaporable compounds used in public room facilities and afterwards sends them to a government database that determines whether the particles are of interest.

Still yet another advantage of the present invention is that provides a system that uses artificial intelligence to update itself when new sounds or particles are detected by the detector.

While the inventor's description contains many specificities, these should not be construed as limitations of the environment monitoring and control system used to monitor public closed room facilities, but rather as an exemplification of several preferred embodiments thereof, any other variations may be possible. Accordingly, the scope should be determined not by the embodiments illustrated, but by the specification, the drawings, and the claims and any legal equivalent thereof.

What is claimed is:

1. An environment monitoring and control system used to monitor a closed room facility for the detection of a public nuisance or a gas particle that can be identified as a controlled substance, the system comprises:
a detector that has particle sensors, an array of chemical sensors, a motion sensor, a sound sensor, a detector communication device, a detector processor that is configured to record any particle, chemical, movement, or sound sensed, hereinafter collectively referred as a recorded value, by either the particle sensor, the array of chemical sensors, the motion sensor, or sound sensor and to transmit the recorded value to either to a personal handheld device or to a central processing system via the detector communication device of the detector, the detector is configured to be placed in the closed room facility;
the central processing system that is configured to interpret the recorded value received from the detector, a communication module that is configured to communicate with either the detector, a personal handheld device, a local police authority, or a governmental database, the central processing system has a central processor database, the central processing system is configured to use the central processor database to interpret the recorded value received from the detector and to determine if the recorded value is of interest, the central processor database has a plurality of known values for sounds, chemicals, particles, and movement that are deemed of interest, if the recorded value is deemed of interest, then the central processing system is configured to send an alarm either to the detector, to the personal handheld device, to the local police authority, or to the governmental database, the central processing system has an artificial intelligence module that is configured to determine if the recorded value is new, and if so, then to add the recorded value to the central processor database, the artificial intelligence module is also configured to scour an internet to determine if the recorded value is of interest, and if so, then to identify the recorded value as a value that is of interest; and
a personal handheld device that has a handheld device processor and a transceiver, the handheld device is configured to communicate with the detector and the central processing system.

2. The environment monitoring and control system used to monitor the closed room facility for the detection of the public nuisance or the gas particle that can be identified as the controlled substance of claim 1, the system comprises the governmental database that is configured to receive the recorded value, if it is new, and to send the recorded value to a governmental monitoring system that is configured to determine if the recorded value is of interest, if so, then to update the governmental database with the governmental value and to send the central processing system with a new value deemed of interest.

3. The environment monitoring and control system used to monitor the closed room facility for the detection of the public nuisance or the gas particle that can be identified as the controlled substance of claim 1, the system comprises the local police authority, the local police authority has a communication module that is configured to receive alarms from either the central processing system or the detector.

4. The environment monitoring and control system used to monitor the closed room facility for the detection of the public nuisance or the gas particle that can be identified as the controlled substance of claim 1, wherein the detector comprises of a humidity sensor and a temperature sensor, the detector is configured to transmit any value collected from either the humidity sensor or the temperature sensor to the central processing system.

5. The environment monitoring and control system used to monitor the closed room facility for the detection of the public nuisance or the gas particle that can be identified as the controlled substance of claim 1, wherein the detector comprises a vibration sensor, the detector is configured to transmit any value collected from the vibration sensor to the central processing system.

6. The environment monitoring and control system used to monitor the closed room facility for the detection of the public nuisance or the gas particle that can be identified as the controlled substance of claim 1, wherein the detector comprises of a detector module that is configured to store values that correspond to a value deemed of interest and to transmit the value deemed of interest to either the personal handheld device, the local police authority, or the central processing system.
